(19) United States
(12) Patent Application Publication
(10) Pub. No.: US 2020/0054794 A1
(43) Pub. Date: Feb. 20, 2020

(54) Biodegradable Wire Implant

(71) Applicant: Syntellix AG, Hannover (DE)

(72) Inventors: Jan Marten Seitz, Hannover (DE); Katrin Nagel, Hannover (DE)

(73) Assignee: Syntelliz AG

(21) Appl. No.: 16/540,910

(22) Filed: Aug. 14, 2019

(30) Foreign Application Priority Data
Aug. 17, 2018 (DE) 10 2018 120 093

(51) Int. Cl. A61L 27/58; A61L 27/04

(52) U.S. Cl. CPC A61L 27/58; A61L 27/047; A61L 27/30; A61L 2300/412; A61L 2430/02

(57) ABSTRACT

The invention relates to a wire implant, in particular for wire osteosynthesis, and a corresponding method for its production. The wire implant has been subjected to a heat treatment, wherein the wire implant consists of a biocompatible, biocorrodible magnesium alloy, which is composed of metallic magnesium of at least 80 wt. % a zinc proportion of 0.1 to 2.0 wt. %, a zirconium proportion of 0.1 to 2.0 wt. %, a proportion of rare earth metals of 0.1 to 10 wt. %, wherein the yttrium content among the rare earth metal content proportion is 0.1 to 5.0 wt. %, a manganese proportion of 0.01 to 0.2 wt. %, an aluminium proportion of less than 0.1 wt. %, a proportion of copper, nickel and iron of less than 0.10 wt. % in each case, and a proportion of other physiologically undesirable impurities totaling less than 0.8 wt. %, wherein the remainder of the alloy is magnesium up to 100 wt. %.

### FIELD OF THE INVENTION

The invention relates to a wire implant, in particular for wire osteosynthesis, and a corresponding method for its production.

### Prior Art

When a bone fracture occurs, an osteosynthesis procedure is often necessary to connect the bone fragments belonging to each other in an anatomically correct position and fix them in place. Depending on the type of fracture, different fixation means and procedures are used.

In wire osteosynthesis, wire implants are used to connect and fix the bone fragments.

In spiked wire osteosynthesis, the bone fragments are fixed after reduction by means of so-called spiked or Kirschner wires (K-wire). These drill wires are usually made of stainless steel or titanium and are drilled into the bone by rotation. Kirschner wire osteosynthesis goes back to its inventor and developer, Dr Martin Kirschner (1879-1942). The advantage of osteosynthesis with wires is that they can be made via relatively small skin incisions. After completion of bone fracture healing, the wires have to be removed again.

Wire implants can also be used to wrap around bone fragments and in this way connect and fix them (wire cerclage).

Removal of the wires is usually done under general anaesthesia, which always entails a certain risk of anaesthesia. In addition, after the removal of K-wires made of stainless steel or titanium, small openings and cavities in the bone always and inevitably remain, which must heal again.

In order to avoid the disadvantages of implant removal, recently bioabsorbable implants for osteosynthesis have been used more and more frequently. They do not have to be surgically removed after healing, as they dissolve in the body and sometimes are even converted to bone matter.

WO 2015/137911 A1 describes an orthopaedic fastening device which may be designed as a K-wire and is coated with a degradable hydrogel. After implantation of the fastening device, the hydrogel increases in volume and thus contributes to the internal fixation of the fracture. The hydrogel is degraded over time. The implant can be made of magnesium.

WO 2017/035072 A1 describes degradable, magnesium-based implants for bone fixation. The magnesium alloy can also be used as a wire that is wound around bone for fixation (wire cerclage) or as a pin.

### Problem Addressed

The aim of the invention is to provide a degradable wire implant which provides high strength with simultaneously high elasticity.

Biodegradable wire implants require some special mechanical properties of the resorbable metal, which may be less important for other implants such as screws, nails, plates or splints. In particular, high rigidity and high strength of the wire are necessary. Otherwise, the K-wire would warp, twist or kink when screwing into the bone.

### Solution

This problem is addressed by the subject matter of the independent claims. Advantageous further developments of the subjects of the independent claims are characterised in the dependent claims. The wording of all the claims is used in reference to the content of this description. The use of the singular is not meant to exclude the plural, and also vice versa, unless otherwise disclosed.

To address the problem, a wire implant, in particular for wire osteosynthesis, is proposed for positioning and/or fixing at least one broken or osteotomized bone, wherein the wire implant has undergone a heat treatment,

and wherein the wire implant consists of a biocompatible, biocorrodible magnesium alloy composed of metallic magnesium of at least 80 wt. %, a zinc proportion of 0.1 to 2.0 wt. %, a zirconium proportion of 0.1 to 2.0 wt. %, a proportion of rare earth metals of 0.1 to 10 wt. %, wherein the yttrium content among the rare earth metal content proportion is 0.1 to 5.0 wt. %, a manganese proportion of 0.01 to 0.2 wt. %, an aluminium proportion of less than 0.1 wt. %, a copper, nickel and iron proportion in each case of less than 0.1 wt. %, and a proportion of other physiologically undesirable impurities totaling less than 0.8 wt. %, wherein the remainder of the alloy is magnesium up to 100 wt. %.

The term wire implant includes all wires, such as spiked or Kirschner wires or wire cerclages that can be used for osteosynthesis.

Preferably, the magnesium-based alloy contains at least 88 wt. % of magnesium, 0.10 to 1.00 wt. % of zirconium, 0.01 to 1.00 wt. % of zinc, 1.00 to 3.00 wt. % of yttrium and 2.00 to 5.00 wt. % of other rare earth metals. It is preferred that the magnesium-based alloy has an overall content of physiologically undesirable impurities of the metals iron, copper, nickel and aluminium of less than 0.02 wt. %, in relation to the alloy. In particular, the magnesium-based alloy contains less than 0.01 wt. % of aluminium, less than 0.20 wt. % of iron, less than 0.20 wt. % of manganese and less than 0.02 wt. % in each case of copper and nickel. Furthermore, it is preferred that the magnesium-based alloy contains less than 0.01 wt. % of aluminium, less than 0.20 wt. % of zinc, less than 0.15 wt. % of manganese, less than 0.20 wt. % of lithium, less than 0.01 wt. % silicon, less than 0.01 wt. % of iron, less than 0.03 wt. % copper and less than 0.005 wt. % of nickel.

The term “unavoidable other impurities” means elements which, according to general medical experience, cannot be expected to have any adverse physiological effects and cannot be completely ruled out due to the lack of 100% purity of the starting materials. Examples are carbon, silicon, sodium, potassium, oxygen, nitrogen, hydrogen, calcium.

According to the invention, however, the wires must additionally undergo a heat treatment in order to achieve sufficient torsional strength for long, thin knitting needle-shaped implants.

In one embodiment, the wire implant is subjected to heat treatment by solution annealing, quenching and subsequent artificial aging. Likewise, soft annealing is possible. This list is not exhaustive. Other methods of heat treatment are also possible for a person skilled in the art.

The wires or wire sections treated by heat treatments are malleable and bendable. In addition, heat treatment allows the degradation behaviour of the wire to be made different in individual sections. In this way it is possible to provide a wire implant which is customised to the patient or to the necessary treatment.

The basic mechanical properties of the wire implant are therefore determined by the composition of the magnesium alloy as well as the subsequent heat treatment, or by the interaction of these factors.

By means of the heat treatment, on the one hand, in the heat-treated region, the values of the yield strength or tensile strength can be significantly lowered and the elongation at break increased. The wires or wire sections treated by heat treatments are thus malleable and bendable. On the other hand, particularly strong regions in terms of torsion and bending can also be produced by the heat treatment.

Soft annealing significantly lowers the values of the yield strength, and the elongation at break increases significantly. This leads in practice to good malleability and bendability of the wire at the sites thus treated.

Aging achieves in particular high strengths, which in particular improve the tensile and torsional strength of the wire at the sites treated in this way.

Wires comprising segments of different strength or ductility have the advantage that, for example tribologically stressed parts of an implant, such as the tip intended for drilling, can be made particularly strong and thus hard, so the cutting work and the attrition/abrasion occurring during implantation are kept low. At the same time, the subsequent more ductile portion of the wire allows moulding to the area of operation and even allows the formation of a cerclage.

Furthermore, the segments of the wire resulting from the different heat treatments can have different degradation rates, by means of which, for example, the influences of areas of different tissues on the degradation behaviour of the magnesium alloy used can be compensated in a targeted manner. For example, the implant can be designed such that the portion of wire remaining in the soft tissue has a much lower rate of degradation than that in the bone. Since the degradation of magnesium in the soft tissue is faster because of the higher water content and the higher substance transport rate, heat treatment which results in a low degradation rate would be advantageous for the wire segment used here. In principle, in this way the degradation progress can also be controlled such that the wire dissolves in a targeted manner from its ends and not evenly over its entire surface. This can carry the advantage that the implant remains intact for a particularly long time at critical points.

In addition, the heat treatments can be used to make the degradation behaviour of the wire different in individual sections. Using heat treatment, degradation rates of 0.05 to 2.0 mm per year are possible in a heat-treated region.

In this way it is possible to provide a wire implant which is tailored to the patient or to the necessary treatment.

In one embodiment, the wire implant is heat-treated over its entire length. However, it may be desirable to produce a certain deformability and flexibility in only one subsection or a plurality of subsections of the wire in order to be able to press displaced, strung-up bone fragments into the anatomically correct position. In a further embodiment, the heat treatment takes place in at least one subsection. In particular, heat treatment in about the middle third is advantageous, preferably in one third to two thirds of the total length of the wire implant.

The different possibilities of heat treatment such as aging and soft annealing can also be applied to a wire. For example, a wire having a high-strength tip can be produced, while the remainder of the wire is softer and more flexible.

The heat treatment can take place over the entire length of the wire implant or in at least one subsection. In one embodiment, the degradable wire implant is heat-treated over its entire length. However, it may be desirable to produce a certain deformability and flexibility in only one subsection or a plurality of subsections of the wire in order to be able to press displaced, strungup bone fragments into the anatomically correct position. In particular, a heat treatment in about the middle third is advantageous. The term subsection in the sense of this invention encompasses at least a region of 1% of the total length of the finished implant.

Furthermore, the elongation at break in a soft-annealed region is at least 18%, preferably at least 20%. The elongation at break in a region subjected to aging is at most 3.5%.

In another embodiment, the yield strength in region subjected to aging is at least 360 MPa, preferably at least 380 MPa. In addition, it is advantageous if the yield strength in a soft-annealed region is at least 240 MPa.

It is favourable if the tensile strength in a soft-annealed region is at least 390 MPa.

The diameter of the wire implant is 0.2 mm to 6.0 mm, preferably 0.5 mm to 4.0 mm, and the length of the wire implant is 30 mm to 600 mm, preferably 50 mm to 500 mm.

The final diameter, shape and form of the degradable wire implants are preferably produced from rod-shaped, semi-finished products by cold or hot working, i.e. by the well-known methods of rolling, extrusion and wire drawing. This list is not exhaustive. Other methods are also conceivable for a person skilled in the art.

It is favourable if the wire is round or polygonal or has longitudinal grooves. Also, threadlike depressions are possible. Instead of round wires, it is also possible to produce polygonal wires, for example triangular or square, or fluted wires having longitudinal grooves. These can be produced by extrusion or drawing using suitable matrices. It is also conceivable for the shape of the wire to be changed after its production by means of suitable methods, for example by means of milling.

The ends of the degradable wire implant can be flat or pointed. It is advantageous for the degradable wire implant, in particular a K-wire, to have a tip at at least one of the ends. The tip can be designed for example as a trocar, lancet, chisel tip or as a drill neck. The common trocar or lance-shaped tips can be added by milling, grinding, cutting or moulding. A loop-shaped hole is present at least at one of the ends of the degradable wire implant, in particular a K-wire.

In a further embodiment, the wire implant has a smooth surface in at least one subsection, preferably a smoothly polished surface.

It is favourable if the wire implant has a roughness depth of less than 1.0 μm, preferably of 0.8 μm, in at least one subsection. The roughness is measured as a Rz value (mean roughness depth). The roughness depth can be achieved, for example, by mechanical polishing, by oscillating cross-grinding, so-called superfinishing. Also, special methods of electrochemical polishing can achieve this low degree of roughness. The low degree of roughness achieves extended degradation time or a reduced degradation rate.

It has been found that a roughness depth of less than 1.0 μm leads to a delay in the degradation by 100 to 200 hours.

Furthermore, the wire implant is hollow in at least one subsection. One or more substances, preferably medications, can preferably be embedded in the cavity so that there is the possibility of postoperative pharmaceutical treatment of the patient. As the implant degrades, this integrated drug depot becomes accessible over time, and delivery of the drug to the surrounding endogenous bone and soft tissue becomes possible. It is also possible to use a substance in the cavity which by its release can determine the degradation rate.

The aim is further achieved by a method for producing a wire implant, wherein the wire implant is subjected to a heat treatment over the entire length of the wire or in at least a subsection of the wire implant, and wherein the wire implant consists of a biocompatible, biocorrodible magnesium alloy composed of metallic magnesium of at least 80 wt. %, a zinc proportion of 0.1 to 2.0 wt. %, a zirconium proportion of 0.1 to 2.0 wt. %, a proportion of rare earth metals of 0.1 to 10 wt. %, wherein the yttrium content among the rare earth metal content proportion is 0.1 to 5.0 wt. %, a manganese proportion of 0.01 to 0.2 wt. %, an aluminium proportion of less than 0.1 wt. %, a proportion of copper, nickel and iron of less than 0.10 wt. % in each case, and a proportion of other physiologically undesirable impurities totaling less than 0.8 wt. %, wherein the remainder of the alloy is magnesium up to 100 wt. %.

To produce the biodegradable wire implant, preferably a magnesium alloy is first produced, which is advantageously composed of metallic magnesium of at least 80 wt. %, a zinc proportion of 0.1 to 2.0 wt. %, a zirconium proportion of 0.1 to 2.0 wt. %, a proportion of rare earth metals of 0.1 to 10 wt. %, wherein the yttrium content among the rare earth metal content proportion is 0.1 to 5.0 wt. %, a manganese proportion of 0.01 to 0.2 wt. %, an aluminium proportion of less than 0.1 wt. %, a copper, nickel and iron proportion in each case of less than 0.1 wt. %, and a proportion of other physiologically undesirable impurities totaling less than 0.8 wt. %, wherein the remainder of the alloy is magnesium up to 100 wt. %.

By means of suitable common methods of cold or hot forming, such as extrusion, a method of cold and hot pressing or compacting, forging or rolling or other methods of cold or hot forming, a magnesium moulding is preferably made.

A moulded part produced in this way is then further processed to the desired wire implant. The final diameter, form and shape of the wire implants are preferably produced from semi-finished products by cold or hot working, in particular by the well-known methods of rolling, extrusion and wire drawing. This list is not exhaustive. Other methods are also conceivable for a person skilled in the art.

The heat treatment of the wire implant takes place over the entire length of the wire or in at least one subsection.

For the purposes of this invention, heat treatment is understood to mean treatment of the wire implant in one or more steps, wherein the wire implant is heated and cooled again in a certain time pattern in order to change material properties.

By the heat treatment, in particular by solution annealing, quenching and subsequent artificial aging or by soft annealing, the wires are given sufficient torsional strength and bending strength for long, thin and knitting needle-shaped components.

In one embodiment, the wire implant is subjected to heat treatment by solution annealing, quenching and subsequent artificial aging. Likewise, soft annealing is possible. This list is not exhaustive. Other methods of heat treatment are also possible for a person skilled in the art.

Solution annealing is preferably carried out at a temperature of from 300° C. to 550° C., preferably from 350° C. to 500° C., more preferably at 480° C. The solution annealing is preferably carried out over a period of 2 to 100 minutes, particularly preferably 3 to 60 minutes. The quenching is preferably carried out in water, oil or cold air. Artificial aging is preferably carried out at 120 to 250° C., particularly preferably at 180° C. over a period of 2 h-48 h.

The solution annealing is carried out in a contactless manner by laser light or by inductive heating or by heating by means of infrared irradiation or in an oven, for example a muffle furnace or a tube furnace, preferably under protective gas or in a (partial) vacuum.

In another embodiment, the wire implant is subjected to soft annealing as a form of heat treatment. Preferably, the soft annealing is carried out at 350° C. to 420° C., particularly preferably at 400° C., over a period of 5 to 60 minutes. If the entire wire implant is soft-annealed, the strength decreases, but the wire becomes deformable. If only a section of the wire implant is to become soft, the region that is to remain tough and stiff, such as the trocar tip, must be cooled during the procedure, or it must not be in the heat treatment zone of influence.

By means of the heat treatment, on the one hand, in the heat-treated region, the values of the yield strength or tensile strength can be significantly lowered and the elongation at break increased. The wires or wire sections treated by heat treatments are thus malleable and bendable. On the other hand, particularly strong regions in terms of torsion and bending can also be produced by the heat treatment.

Soft annealing significantly lowers the values of the yield strength, and the elongation at break increases significantly. This leads in practice to good malleability and bendability of the wire at the sites thus treated.

Aging achieves in particular high strengths, which in particular improve the tensile and torsional strength of the wire at the sites treated in this way.

In addition, the heat treatments can be used to make the degradation behaviour of the wire different in individual sections. Using heat treatment, degradation rates of 0.05 to 2.0 mm per year are possible in a heat-treated region.

In one embodiment, the wire implant is heat-treated over its entire length. However, it may be desirable to produce a certain deformability and flexibility in only one subsection or a plurality of subsections of the wire in order to be able to press displaced, strung-up bone fragments into the anatomically correct position. In a further embodiment, the heat treatment takes place in at least one subsection. In particular, heat treatment in about the middle third is advantageous, preferably in one third to two thirds of the total length of the wire implant.

The different possibilities of heat treatment such as aging and soft annealing can also be applied to a wire. For example, a wire having a high-strength tip can be produced, while the remainder of the wire is softer and more flexible.

Furthermore, it is advantageous if the wire implant has a smooth surface, preferably a smoothly polished surface, in at least one subsection. It has been found that very smoothly polished surfaces of the degradable wire implants result in the almost always desired delay in the onset of degradation. The low roughness depth can extend over the entire length of the wire or in at least one subsection. It has been found that a roughness depth of less than 1.0 μm leads to a delay in degradation of 100 to 200 hours.

It is favourable if the wire implant has a roughness depth of less than 1.0 μm, preferably of 0.8 μm, in at least one subsection. The roughness is measured as the Rz value (average roughness depth). The roughness depth can be achieved, for example, by mechanical polishing, by oscillating cross-grinding, so-called superfinishing. This low degree of roughness can also be achieved by special methods of electrochemical polishing or lapping. Low roughness achieves an extended degradation time or a reduced degradation rate.

Another subject matter of the present invention relates to a wire implant which is obtainable by the method described above.

Further details and features arise from the following description of preferred exemplary embodiments in conjunction with the dependent claims. In this case, the features can be implemented individually in combination. The possibilities for solving the problem are not limited to the exemplary embodiments. For example, specified ranges always includes all intermediate values and all imaginable subranges which are not mentioned.

The exemplary embodiments are shown schematically in the figures. The same reference signs in the individual figures designate the same or functionally identical elements or elements which correspond to one another in respect of their functions. Specifically, the figures show the following:

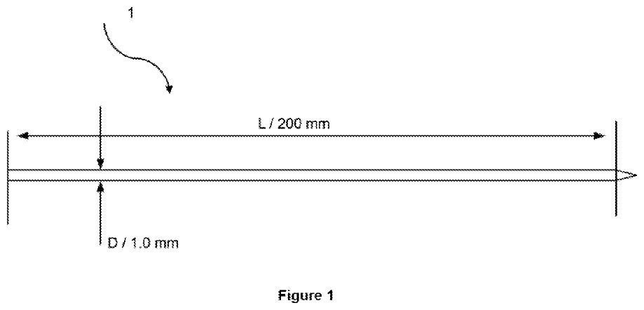

FIG. 1 shows a biodegradable wire implant.

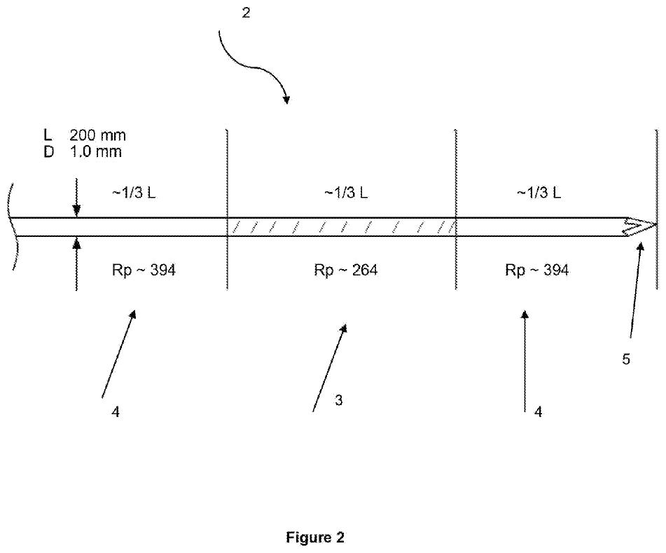

FIG. 2 shows a biodegradable wire implant having a heat-treated section and a trocar tip.

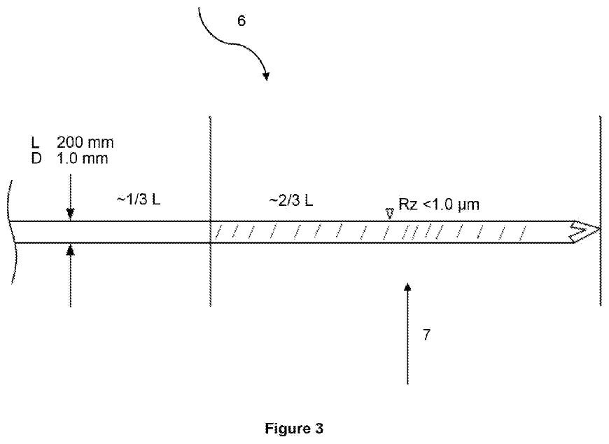

FIG. 3 shows a biodegradable wire implant having polished sections.

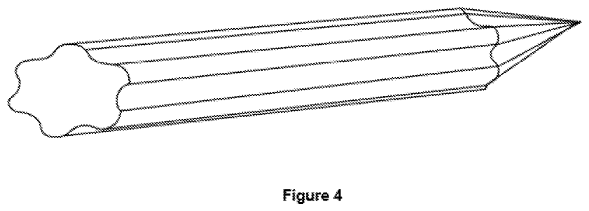

FIG. 4 shows a biodegradable wire implant having a hexagonal outer profile.

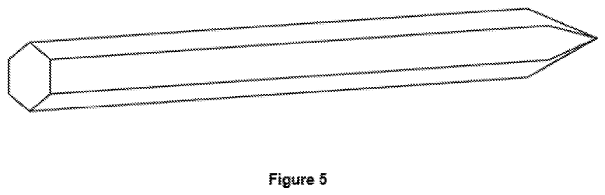

FIG. 5 shows a biodegradable wire implant having a fluted profile.

FIG. 1 shows a schematic representation of the degradable wire implant (1). To produce the biodegradable wire implant, a magnesium alloy comprising the following components is preferably first produced:

Rare earths: 8.4 wt. %
(of which neodymium: 2.1 wt. %

Yttrium: 1.6 wt. %

Zirconium: 0.4 wt. %

Zinc: 0.6 wt. %

Traceable Impurities:

Iron: 0.013 wt. %

Copper: 0.036 wt. %

Nickel: 0.003 wt. %

Aluminium: 0.0032 wt. %

Lithium: 0.0035 wt. %

The remainder is magnesium.

A fabricated magnesium moulded part is further processed into the desired wire implant.

By means of cold wire drawing, a wire of 1.0-mm diameter (D) and 60-cm length (L) was first produced. This was cut into 3 equal pieces of 200-mm length (L) to demonstrate the effect of heat treatment (1). Wire 1 was left in its original, drawn state. The mechanical strength values are shown in table 1, column 1. Wire 2 was solution-annealed at 490° C.+/−10° C. in an argon atmosphere for 1 h, quenched in cold water, then aged at 180° C. for 48 h. The values of the mechanical strength of the K wire thus produced are shown in table 1, column 2. Wire 3 was annealed in a laboratory muffle furnace under argon shielding gas at 400° C. for 60 minutes and cooled very slowly, for about 8 hours, in the oven to RT. The mechanical strength values are shown in table 1, column 3. The same experiment was carried out with a wire of 0.8-mm diameter and 60-cm length. The results are shown in table 2.

Tables 1 and 2 give values of tensile strength (in MPa=N/mm²), yield strength (in MPa=N/mm²) and elongation at break (in % of length) for degradable wire implants in the cold-drawn, aged and soft-annealed states. In each case the mean value of three measurements was given.

TABLE 1
Strength properties of degradable wire implants produced according
to the invention after different heat treatments/diameter 1 mm

| | Wire 1 | Wire 2 | Wire 3 |
|---|---|---|---|
| 1 mm diameter | Cold-drawn | Aged | Soft-annealed |
| Tensile strength Rm, MPa | 360 | 402 | 281 |
| Yield strength Rp (MPa) | 303 | 381 | 262 |
| Elongation at break εB (%) | 9.7 | 2.2 | 21.3 |
| State according to DIN EN 515 | F | T6 | 0 |

TABLE 2
Strength properties of degradable wire implants produced according
to the invention after different heat treatments/diameter 0.8 mm

| | Wire 1 | Wire 2 | Wire 3 |
|---|---|---|---|
| 0.8-mm diameter | Cold-drawn | Aged | Soft-annealed |
| Tensile strength Rm, MPa | 372 | 432 | 287 |
| Yield strength Rp (MPa) | 332 | 412 | 269 |
| Elongation at break εB (%) | 4.3 | 2.1 | 22.4 |
| State according to DIN EN 515 | F | T6 | 0 |

Soft annealing significantly lowers the values of the yield strength, and the elongation at break increases significantly. This leads in practice to good malleability and bendability of the wire at the sites thus treated.

Aging achieves in particular high strengths, which in particular improve the tensile and torsional strength of the wire at the sites treated in this way.

The different possibilities of heat treatment such as aging and soft annealing can also be applied to a wire. For example, a wire having a high-strength tip can be produced, while the remainder of the wire is softer and more flexible.

FIG. 2 shows a biodegradable wire implant (2), which was heat-treated in the middle third (3) by soft annealing. A wire of 1.0-mm diameter (D) was first produced by cold wire drawing, and then cut to a 200-mm length (L).

The yield strength in this heat-treated section is 264 MPa, and 394 MPa in the two non-heat-treated sections in the front third and back third (4). The heat treatment in this subsection of the implant makes it possible to maintain unchanged hardness and toughness of the biodegradable K-wire in the region of the trocar tip (5), and keep it malleable and bendable in the middle region, and hard and tough in the rear region, the guide region.

In addition to the heat treatment, the degradable wire implant can be continuously or sectionally varied in its corrosion rate and adapted to the medical case by polishing or by oxidising and polishing the surface. FIG. 3 shows such a wire (6). This was heat-treated in the middle third by aging, and additionally polished over the front two thirds (7) of the length by means of oscillating grinding to provide a roughness (Rz, average roughness depth) of <1 μm.

Instead of round wires, polygonal wires, such as triangular or tetragonal wires, or cannulated wires with longitudinal grooves can also be produced by extrusion or drawing by means of suitable matrices. FIG. 4 shows a biodegradable wire implant having a hexagonal outer profile; FIG. 5 shows a biodegradable wire implant having a fluted profile.

Numerous modifications and developments of the described exemplary embodiments can be realised.

### REFERENCE SIGNS

1. Biodegradable wire implant
2. Heat-treated, biodegradable wire implant
3. Heat-treated region in the middle third
4. Non-heat-treated section
5. Trocar tip
6. Smoothly polished, biodegradable wire implant
7. Section with roughness (Rz, average roughness depth) of <1 μm
D Diameter
L Length
Rp Yield strength (MPa)
Rz Roughness depth (μm)

### CITED LITERATURE

### Cited Patent Literature

### WO 2015/137911 A1

### WO 2017/035072 A1

### EP 2 753 373 B1

1. A wire implant, in particular for spiked wire osteosynthesis,
wherein
the wire implant has undergone a heat treatment,
and the wire implant consists of a biocompatible, biocorrodible magnesium alloy composed of metallic magnesium of at least 80 wt. %, a zinc proportion of 0.1 to 2.0 wt. %, a zirconium proportion of 0.1 to 2.0 wt. %, a proportion of rare earth metals of 0.1 to 10 wt. %, wherein the yttrium content among the rare earth metal content proportion is 0.1 to 5.0 wt. %, a manganese proportion of 0.01 to 0.2 wt. %, an aluminium proportion of less than 0.1 wt. %, a proportion of copper, nickel and iron of less than 0.10 wt. % in each case, and a proportion of other physiologically undesirable impurities totaling less than 0.8 wt. %, wherein the remainder of the alloy is magnesium up to 100 wt. %.

2. The wire implant as claimed in claim 1,
wherein
the heat treatment takes place over the entire length of the wire or in at least one subsection.

3. The wire implant as claimed in claim 1, further including an elongation at a break in a soft-annealed region of the wire,
wherein
the elongation at break in the soft-annealed region is at least 18%, preferably at least 20%.

4. The wire implant as claimed in claim 3,
wherein
the elongation at break in a region subjected to aging is at most 3.5%.

5. The wire implant as claimed in claim 1,
wherein a
yield strength in a region subjected to aging is at least 360 MPa, preferably at least 380 MPa.

6. The wire implant as claimed in claim 5,
wherein
the yield strength in a soft-annealed region is at least 240 MPa.

7. The wire implant as claimed in claim 1,
wherein a
tensile strength in a soft-annealed region is at least 390 MPa.

8. The wire implant as claimed in claim 1,
wherein
the wire implant is round or polygonal or has longitudinal grooves.

9. The wire implant as claimed in claim 1,
wherein
the wire implant has a diameter of 0.2 mm to 6.0 mm, preferably 0.5 mm to 4.0 mm and the length is 30 mm to 600 mm, preferably 50 mm to 500 mm.

10. The wire implant as claimed in claim 1,
wherein
the wire implant has a smoothly polished surface in at least one subsection.

11. The wire implant as claimed in claim 1,
wherein
the wire implant has a roughness depth of less than 1.0 μm, preferably of 0.8 μm, in at least one subsection.

12. The wire implant as claimed in claim 1,
wherein
the wire implant is hollow in at least one subsection.

13. A method for producing a wire implant as claimed in claim 1,
wherein
the wire implant is heat-treated over the entire length of the wire or in at least one subsection of the wire implant,
and the wire implant consists of a biocompatible, biocorrodible magnesium alloy composed of metallic magnesium of at least 80 wt. %, a zinc proportion of 0.1 to 2.0 wt. %, a zirconium proportion of 0.1 to 2.0 wt. %, a proportion of rare earth metals of 0.1 to 10 wt. %, wherein the yttrium content among the rare earth metal content proportion is 0.1 to 5.0 wt. %, a manganese proportion of 0.01 to 0.2 wt. %, an aluminium proportion of less than 0.1 wt. %, a proportion of copper, nickel and iron of less than 0.10 wt. % in each case, and a proportion of other physiologically undesirable impurities totaling less than 0.8 wt. %, wherein the remainder of the alloy is magnesium up to 100 wt. %.

14. The method for producing a wire implant as claimed in claim 13,
wherein
solution annealing is carried out in a contactless manner by laser light or by inductive heating or by heating by means of infrared irradiation or in a standard muffle furnace.

15. The method for producing a wire implant as claimed in claim 13,
wherein
solution annealing is carried out at a temperature of 300° C. to 520° C., preferably from 350° C. to 500° C., particularly preferably at 480° C.

16. The method for producing a wire implant as claimed in claim 13,
wherein
solution annealing takes place over a period of 2 to 100 minutes, particularly preferably 3 to 60 minutes.

17. The method for producing a wire implant as claimed in claim 1,
wherein
low roughness of the implant surface is achieved by oscillating cross-grinding, mechanical grinding, polishing, lapping or by electrochemical polishing.

18. A wire implant obtainable by a method as claimed in claim 13.
